(19) United States
(12) Patent Application Publication
(10) Pub. No.: US 2026/0235860 A1
(43) Pub. Date: Aug. 13, 2026

(54) STEREOSCOPIC IMAGES FROM MULTIPLE PERSPECTIVES

(71) Applicant: Carl Zeiss Meditec AG, Jena (DE)

(72) Inventors: Johannes Rangel, Aalen (DE); Artur Hoegele, Oberkochen (DE); Gerald Panitz, Bopfingen (DE); Stefan Saur, Aalen (DE); Christoph Hauger, Aalen (DE)

(21) Appl. No.: 19/533,108

(22) Filed: Feb. 7, 2026

(30) Foreign Application Priority Data
Feb. 7, 2025 (DE) 10 2025 104 592.5

(51) Int. Cl. G02B 21/22; A61B 90/00; G02B 21/00

(52) U.S. Cl. CPC G02B 21/22; A61B 90/361; A61B 90/37; G02B 21/0012; A61B 2090/371

(57) ABSTRACT

A surgical visualization system includes three optical channels. A first optical channel forms a stereo pair with a second optical channel and a further stereo pair with a third optical channel. A partially stereoscopic image is generated for the second stereo pair.

### CROSS REFERENCE TO RELATED APPLICATIONS

This application claims priority to German patent application DE 10 2025 104 592.5, filed Feb. 7, 2025, the entire content of which is incorporated herein by reference.

### TECHNICAL FIELD

Various aspects of the disclosure relate to a surgical visualization system and to a method for operating a surgical visualization system. Various aspects of the disclosure relate in particular to a surgical visualization system and to the generation of multiple stereoscopic images which image a surgical site from different perspectives.

### BACKGROUND

In surgical procedures, several surgeons (e.g., a lead surgeon and an assistant surgeon) often work simultaneously with a surgical visualization system which provides a magnified optical image representation of a surgical site. In particular, surgical visualization systems providing a stereoscopic image representation of a surgical site are known. For example, see DE 10 2014 108 811 B3, U.S. Pat. No. 7,002,738 B2 or else DE 10 2015 216 569 B3.

In this case, the stereoscopic imaging process is implemented from a specific perspective, i.e., an image plane of the stereoscopic imaging process has a specific orientation in relation to the surgical site.

In principle, should several observers use the surgical visualization system, it is desirable that each observer is shown respective images which present the surgical site from an appropriate perspective that matches the respective observer's actual perspective of the surgical site. For example, it was found empirically that the hand-eye coordination of humans is impaired when a deviation between the respective observer's actual perspective and the perspective of the surgical site depicted in the images captured with the surgical visualization system becomes larger than approx. 10° to 15°. For example, the hands may be moved incorrectly in that case. Hence, it is particularly important that all observers contributing to the surgical procedure, or at least monitoring the latter, are confronted with only a small offset between the actual perspective and the images captured with the surgical visualization system.

For example, if the lead surgeon is at an azimuth angle of 0° with respect to the surgical site (defined in a reference coordinate system in which the XY-plane is oriented parallel to the focal plane, and the azimuth angle specifies the orientation within the XY-plane) and the assistant surgeon is at an azimuth angle of 90°, then the corresponding perspectives of the images should also depict the surgical site from matching azimuth angles of 0° and 90°. For example, this ensures that those objects in the surgical site that appear on the left in the respective stereoscopic image are also in fact arranged to the left in the surgical site, while those objects for example appearing top right in the stereoscopic image are also arranged top right in the surgical site.

A technique for providing multiple stereoscopic images from multiple perspectives lies in the provision of a first optical channel and a second optical channel for the first observer (e.g., the lead surgeon) and of a third optical channel and a fourth optical channel for the second observer (e.g., the assistant surgeon). The first optical channel images a first region of the surgical site, and the second optical channel images a second region of the surgical site. The first region and the second region have an overlap which defines a corresponding stereoscopic image for the “stereo pair” formed by the first optical channel and the second optical channel. In this context, the central ray of a beam path of the first optical channel is incident on the focal plane at a first polar angle (i.e., at a tilt vis-à-vis the normal of the XY-plane or at a tilt vis-à-vis the Z-axis which is perpendicular to the XY-plane), while the central ray of the beam path of the second optical channel is incident on the focal plane at a second polar angle. The first polar angle and the second polar angle differ from each other, and the difference is referred to as stereo angle. Likewise, the third optical channel images a third region in the focal plane, and the fourth optical channel images a fourth region in the focal plane. The third region and the fourth region in turn have an overlap, and so a corresponding stereo region of the stereo pair formed by the third optical channel and the fourth optical channel is formed. In this case, the first optical channel and the second optical channel image the corresponding stereo region from the perspective of the first observer; and the third optical channel and the fourth optical channel image the stereo region from the perspective of the second observer. Techniques allowing the optics of the third and fourth optical channels to be moved vis-à-vis the optics of the first and second optical channel such that it is possible for example to set an azimuth angle offset between the two perspectives are also known. However, such a technique is disadvantageous in that it requires the formation of a total of four optical channels. This brings about high system complexity. Moreover, the corresponding microscope unit of the surgical visualization system becomes large and heavy.

Various techniques are possible for reducing the number of optical channels required.

One technique is described in DE 10 2009 012 897 B4: in that case, use is made of two optical channels that form a stereo pair-this allows a stereoscopic image to be displayed to a first observer; a third optical channel allows the generation of a monoscopic image of the surgical site for a second observer. Thus, using only three optical channels, this allows the generation of a stereoscopic image for one observer and a monoscopic image for another observer using only three optical channels.

However, in some instances it may be desirable to display stereoscopic information about the surgical site to both observers. For example, in a scenario in which the two observers are arranged on different sides of the surgical site and opposite each other (i.e., the two perspectives have an azimuth angle offset of approx. 180°), a stereoscopic image with a perspective matching that of one of the two observers may be captured using two optical channels only, and this image may subsequently be reflected by digital post-processing. The mirror-inverted stereoscopic image obtained in this way then corresponds to the perspective of the other observer. However, in such a scenario, the relative orientation of the perspectives of the two observers to each other is restricted to an offset of the respective azimuth angles of 180°.

For example, DE 10 2015 216 648 B3 describes a system for the stereoscopic visualization of an object region using three imaging beam paths. In this way, a main observer and a co-observer are each provided with an image of the object region with a spatial visual impression and a different perspective by virtue of the stereo basis for the image information displayed to the main observer and image information displayed to the co-observer differing. The corresponding optical systems all image on a joint image sensor; in this case, different optical channels image onto different regions of the image sensor.

### SUMMARY

There is a need for improved techniques for stereoscopic imaging of a surgical site from multiple perspectives. In particular, there is a need for techniques that eliminate or alleviate at least some of the aforementioned limitations and disadvantages. There is a need for compact surgical visualization systems that provide stereoscopic images from multiple perspectives with a relatively high image resolution and low system complexity.

This problem is solved by a surgical visualization system and a method for operating an electronic data processing device described herein.

A surgical visualization system includes a first optical channel. The first optical channel includes a first camera. The surgical visualization system also includes a second optical channel which has a second camera. Moreover, the surgical visualization system includes a third optical channel, with the third optical channel including a third camera. The first optical channel and the second optical channel image a first stereo region in the focal plane in stereoscopic fashion. The first optical channel and the third optical channel image a second stereo region in the focal plane in stereoscopic fashion. The surgical visualization system also includes an electronic data processing device. The electronic data processing device is configured to control at least one display apparatus for displaying a fully stereoscopic image and a partially stereoscopic image. In this case, the fully stereoscopic image represents the first stereo region in stereoscopic fashion, and the partially stereoscopic image represents the second stereo region in stereoscopic fashion. Moreover, the partially stereoscopic image represents a portion of the third region adjoining the stereo region in monoscopic or synthetic-stereoscopic fashion.

For example, the first optical channel may image the first region of the focal plane from a first angle. The second optical channel may for example image a second region of the focal plane from a second angle. The third optical channel may image a third region of the focal plane from a third angle.

The first stereo region is defined here by an overlap between the first region and the second region. In this case, the second stereo region is defined by an overlap between the first region and the third region.

The first optical channel and the second optical channel thus form a first stereo pair; the first optical channel and the third optical channel thus form a second stereo pair. In this case, the first optical channel is part of two stereo pairs, specifically together with the second optical channel on the one hand and together with the third optical channel on the other hand.

Over its entire image field, the fully stereoscopic image provides natively stereoscopic information for a surgical site arranged in the focal plane. In other words, this means that the first stereo region covers the entire image field of the fully stereoscopic image. Corresponding stereoscopic information is obtained optically by the first stereo pair formed by the first optical channel and the second optical channel. Something else holds true for the partially stereoscopic image. The partially stereoscopic image does not have native stereo information from a pair of optical channels, i.e., native stereo information obtained optically, over its entire image field. Instead, the stereo information is limited to the second stereo region which makes up only a part of the image field of the partially stereoscopic image. There is no native stereo information available in those image portions of the partially stereoscopic image that do not image the stereo region. There, a surgical site arranged in the focal plane can be displayed in monoscopic or synthetic-stereoscopic fashion. In this case, a synthetic-stereoscopic representation of the surgical site corresponds to artificial stereo information for the surgical site which was not generated optically but rather by digital post-processing.

Thus, two images that image a surgical site arranged at the focal plane in stereoscopic fashion are generated, but only three optical channels are used in the process.

The first camera may include a first elongate camera sensor, for example with an aspect ratio of 16:9. The second camera may include a second elongate camera sensor, for example with an aspect ratio of 16:9. The third camera may include a third elongate camera sensor, for example with an aspect ratio of 16:9.

Camera images captured with the first camera have a native perspective which is defined by the arrangement or orientation of the camera sensor of the first camera in relation to the surgical site. For example, if a specific rectangular region of the surgical site is imaged on a rectangular camera sensor, then the corresponding camera images have a native perspective: left-right and top-bottom in the camera image correspond to right-left and top-bottom in the rectangular region if this region is imaged accordingly on the camera sensor or if the camera sensor is arranged accordingly. Additionally, the camera images captured with the second camera have a native perspective which is defined by the arrangement of the camera sensor of the second camera in relation to the surgical site. Camera images captured with the third camera have a native perspective which is defined by the arrangement of the camera sensor of the third camera in relation to the surgical site.

It is conceivable that an orientation of the camera sensor of the first camera in relation to the focal plane corresponds to an orientation of the camera sensor of the second camera in relation to the focal plane. For example, the longitudinal axes of the first camera sensor and of the second camera sensor could extend parallel to each other.

At the same time, however, a longitudinal axis of the camera sensor of the third camera may be rotated vis-à-vis the longitudinal axis of the camera sensors of the first and second cameras (for example about an axis of rotation parallel to the Z-axis). For example, the longitudinal axis of the camera sensor of the third camera may have an azimuth angle offset of approx. 90° vis-à-vis the longitudinal axes of the camera sensors of the first camera and of the second camera. In simple terms, the camera sensor of the third camera is thus rotated vis-à-vis the camera sensors of the first camera and the second camera about an axis of rotation perpendicular to the focal plane. This renders different native perspectives possible for the camera images.

The beam path of the first optical channel may illuminate the whole area of the first camera sensor of the first camera. The beam path of the second optical channel may illuminate the whole area of the second camera sensor of the second camera. The beam path of the third optical channel may illuminate the whole area of the third camera sensor of the third camera.

As a result of these techniques described above, the resolution at which the respective camera images are captured can be comparatively high because the full image resolution of each camera is only used for the respective associated optical channel. As a result, the fully stereoscopic image and the partially stereoscopic image may also be provided with a particularly high image resolution.

Then again, the use of different cameras may result in the overlap of the first region and the third region potentially being smaller than the overlap of the first region and the third region on account of a rotation of the respective camera sensors with respect to one another. In other words, this means that the first stereo region may be larger than the second stereo region. There is less stereo information available for the stereo pair formed by the first optical channel and the third optical channel than for the stereo pair formed by the first optical channel and the second optical channel. By using the partially stereoscopic image, which in addition to the stereoscopic second stereo region represents a portion in monoscopic or synthetic-stereoscopic fashion, it is possible to compensate for this inasmuch as the field of view of the partially stereoscopic image is increased.

In an example, a first stereo axis of the first stereo region and a second stereo axis of the second stereo region form an angle in the range from 85° to 95°. In this way, two observers positioned perpendicular to each other may each obtain stereo information with the matching perspective and stereo alignment.

It is possible for a first camera sensor of the first camera to be rectangular and have a first longitudinal axis. A second camera sensor of the second camera may be rectangular with a second longitudinal axis. A third camera sensor of the third camera may be rectangular with a third longitudinal axis. The first longitudinal axis and the third longitudinal axis may form an angle in this case which corresponds to the angle between the first stereo axis and the second stereo axis. Moreover, the second longitudinal axis and the third longitudinal axis may form an angle between them which corresponds to the angle between the first stereo axis and the second stereo axis. In this case, suitable perspectives of the surgical site may be combined with suitable stereo axes in each case.

A ratio of a dimension of a field of view of the fully stereoscopic image to a dimension of a field of view of the partially stereoscopic image may be in the range from 95% to 105%. In other words, this means that the fields of view of the two images may be approximately the same size. As a result, a main observer and a co-observer can see a similarly large section of the surgical site.

A ratio of the first stereo region to the second stereo region cannot be less than 150%. In other words, this means that the first stereo region-for example for the main observer is significantly larger than the second stereo region-for example for the co-observer. Such a variant is helpful in particular when the various camera sensors as discussed above have a 16:9 aspect ratio, with the third camera sensor being oriented at right angles to the first and second camera sensors.

In one variant, the first optical channel can image the first region with a first magnification, and the second optical channel can image the second region with the first magnification as well. The third optical channel can image the third region with a second magnification which is larger than the first magnification. In other words, this means that the first and the second optical channels might image the respective region with the same magnification, but the third optical channel represents the associated third region with a larger magnification factor. As a result, the overlap between the first stereo region and the second stereo region can be relatively large.

For example, the first region, the second region and the third region might all be rectangular and have the same aspect ratio. This enables the use of standardized camera sensors which for example have a 16:9 aspect ratio.

In various examples, the electronic data processing device may be configured to control the at least one display apparatus to display the partially stereoscopic image when a first display mode is activated.

In various examples, the electronic data processing device may be configured to control the at least one display apparatus to display a fully monoscopic image when a second display mode is activated.

The fully monoscopic image may in this case represent the entire third region in monoscopic fashion. Thus, it does not contain any (native or synthetic) stereo information.

Thus, in other words, switching between a partially stereoscopic and a fully monoscopic implementation of the image is conceivable, for example according to user preference. For example, there may be users who prefer a partially stereoscopic representation over a fully monoscopic representation of the surgical site in some phases of a surgical procedure, while the preference may be for a fully monoscopic representation of the surgical site during other phases of a surgical procedure. For example, a partially stereoscopic representation could be preferred if a depth impression is needed to locate specific structures or perform navigation of surgical equipment, for example in a deep channel. By contrast, a fully monoscopic representation could be preferred if the surgical site has little height variation perpendicular to the optical axis, i.e., if it is relatively flat.

In an alternative to switching between a partially stereoscopic display mode and a fully monoscopic display mode or in addition to that, it is also possible to switch between a partially stereoscopic display mode and a fully stereoscopic display mode. Thus, the electronic data processing may be configured to control the at least one display apparatus to display a further fully stereoscopic image when a third display mode is activated. In this case, the further fully stereoscopic image may exclusively represent the second stereo region in stereoscopic fashion. Typically, the field of view of this further fully stereoscopic image will therefore be smaller than the field of view of the partially stereoscopic image.

In this case, the electronic data processing device may be configured in various examples to switch between the monoscopic representation of the synthetic-stereoscopic representation of the at least one portion of the third region adjacent to the stereo region based on an activated mode of operation. Such techniques are based on the insight that some users prefer a monoscopic representation over a synthetic-stereoscopic representation (which may be associated with a certain amount of uncertainty owing to the necessary approximation using an appropriate model).

The synthetic-stereoscopic representation may be generated with a model that receives one or more camera images, for example from the first camera and/or the second camera and/or the third camera. Further contextual information relating to the surgical site may be obtained through the use of multiple camera images, and this allows for a better reconstruction of the stereo information.

In various examples, the electronic data processing device is configured to graphically separate the representation of the second stereo region from the representation of the adjacent portion of the third region. Such graphical separation may provide improved distinguishability for those image portions of the partially stereoscopic image in which native stereo information is available and those image portions of the partially stereoscopic image in which no native stereo information is available. This can instill the observer with confidence in the information displayed in the partially stereoscopic image.

The electronic data processing device may be configured to smooth a transition between a portion of the partially stereoscopic image representing the second stereo region and a portion of the partially stereoscopic image representing the adjacent portion of the third region with an image smoothing algorithm. This can ensure a continuous image impression across the entire image field of the partially stereoscopic image.

A method for operating an electronic data processing device associated with a surgical visualization system includes obtaining a first camera image. The first camera image is obtained from a first camera. The first camera is part of a first optical channel of the surgical visualization system. In this context, the first camera image images a first region of a focal plane from a first perspective. Moreover, a second camera image is also obtained from a second camera of a second optical channel of the surgical visualization system. In this case, this second camera image images a second region of the focal plane, with the second region also being imaged from the first perspective. Furthermore, a third camera image is obtained from a third camera of a third optical channel of the surgical visualization system. The third camera image images a third region of the focal plane from a second perspective, with the second perspective differing from the first perspective. Then, a stereoscopic image is generated based on the first camera image as corresponding first stereo channel and based on the second camera image as corresponding second stereo channel. A portion of the first camera image is cut out and rotated based on a difference between the first perspective and the second perspective in order to obtain a partial camera image. A second stereoscopic image is generated based on the partial camera image as corresponding first stereo channel and based on the third camera image as corresponding second stereo channel. The method includes controlling a display device to display the first stereoscopic image and controlling a further device to display the second stereoscopic image.

By performing image post-processing on the first camera image, it is thus possible to adjust the perspective of the first camera image to the perspective of the third camera image so that these form a stereo pair. However, cutting out and rotating result in the overlap between the region imaged by the first camera image and the region imaged by the third camera image (the corresponding stereo region) being relatively small, and so the portion of the second stereoscopic image containing native stereo information is comparatively small (in particular, it is typically smaller than the stereo region with native stereo information in the first stereoscopic image).

As already described above, the first stereoscopic image may be a fully stereoscopic image. The second stereoscopic image may be generated as a partially stereoscopic image or as a fully stereoscopic image-for example depending on an active display mode. For example, it would be possible to switch between display modes so that a partially stereoscopic image is generated at one point and a fully stereoscopic image is generated at another point. A user may switch between the display modes. The switchover could also be implemented in automated fashion, e.g. depending on the operation progress, visible instruments, etc. It is possible to monitor for the presence of one or more switching criteria.

The first camera image can image the first region with a first magnification, the second camera image can image the second region with the first magnification, and the third camera image can image the third region with a second magnification. Thus, different magnifications for the first and second stereoscopic images can be rendered possible. There could be an interpolation of the corresponding pixels during the post-processing of the first camera image. Alternatively, the third camera image could also be post-processed in order to obtain appropriate matching of the pixel resolution.

The second magnification may be larger than the first magnification.

The second stereoscopic image may be generated as a partially stereoscopic image. The method may furthermore include: smoothing or graphically emphasizing a transition between a portion of the partially stereoscopic image representing native-stereoscopic information and a further portion of the partially stereoscopic image representing monoscopic or synthetic-stereoscopic information. The step of smoothing and/or graphically emphasizing firstly allows a continuous representation of features in the surgical site. Then again, it may give the user an indication as to where native-stereoscopic information is present and where no native-stereoscopic information is present.

The features set out above and features described below can be used not only in the applicable combinations that are explicitly set out, but also in other combinations or in isolation, without departing from the scope of protection of the present disclosure. In particular, it would be possible for example for aspects which were described above in connection with the method for operating an electronic data processing device to be combined with those aspects described in connection with the surgical visualization system.

### BRIEF DESCRIPTION OF THE DRAWINGS

The disclosure will now be described with reference to the drawings wherein:

FIG. 1 schematically illustrates a surgical visualization system according to various exemplary embodiments, having three optical channels for generating two stereoscopic images from different perspectives,

FIG. 2 schematically illustrates the different perspectives,

FIG. 3 and FIG. 4 schematically illustrate the beams of the three optical channels according to respective exemplary embodiments of the disclosure,

FIG. 5 and FIG. 6 schematically illustrate stereo regions which arise from the pairwise overlap of the various optical channels according to various exemplary embodiments of the disclosure,

FIG. 7 schematically illustrates the regions of the surgical sites imaged by the optical channels according to various exemplary embodiments of the disclosure,

FIG. 8 illustrates camera images associated with the three optical channels,

FIG. 9 illustrates a fully stereoscopic image generated by combining two camera images,

FIG. 10 schematically illustrates an image portion of a camera image in a stereo region according to various exemplary embodiments of the disclosure,

FIG. 11 schematically illustrates the image portion from FIG. 10 following a rotation,

FIG. 12 illustrates a partially stereoscopic image generated by combining the rotated image portion from FIG. 11 with a camera image,

FIG. 13 is a flowchart of a method according to an exemplary embodiment of the disclosure,

FIG. 14 illustrates various display modes of the surgical visualization system according to various exemplary embodiments of the disclosure, and

FIG. 15 schematically illustrates an electronic data processing device according to various exemplary embodiments of the disclosure.

### DESCRIPTION OF EXEMPLARY EMBODIMENTS

The above-described properties, features and advantages of this disclosure and the way in which they are achieved will become clearer and more clearly understood in the context of the following description of the exemplary embodiments, which are explained in detail in conjunction with the drawings.

The present disclosure is explained in detail below based on preferred embodiments with reference to the drawings. In the figures, identical reference signs designate identical or similar elements. The figures are schematic representations of various embodiments of the disclosure. Elements illustrated in the figures are not necessarily illustrated as true to scale. Rather, the various elements illustrated in the figures are rendered in such a way that their function and general purpose become comprehensible to the person skilled in the art. Connections and couplings between functional units and elements illustrated in the figures can also be implemented as an indirect connection or coupling. A connection or coupling can be implemented in a wired or wireless manner. Functional units can be implemented as hardware, software or a combination of hardware and software.

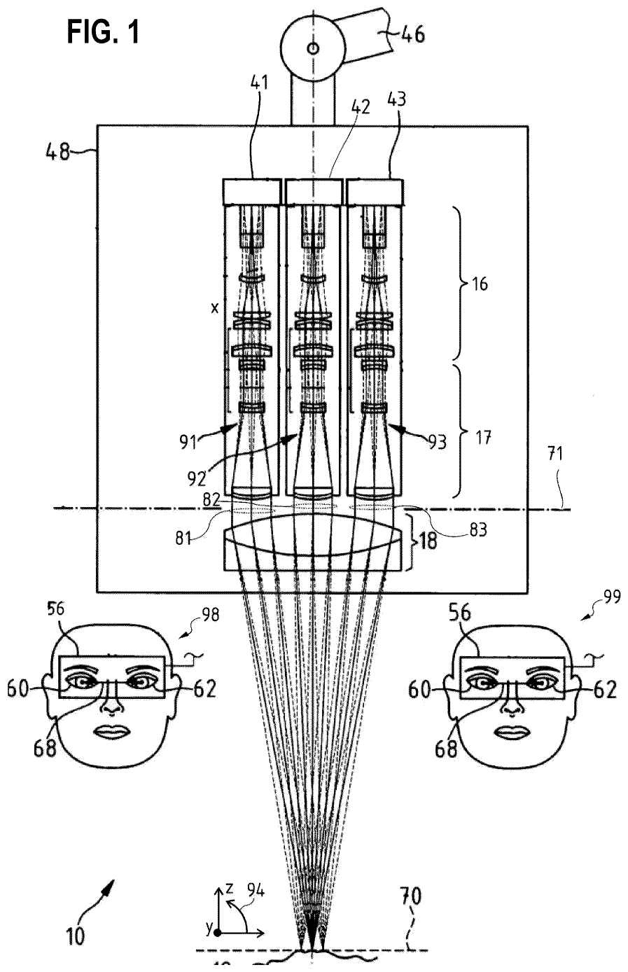

FIG. 1 schematically illustrates a surgical visualization system 10, which is configured for the provision of a microscopic image representation of a surgical site 12. The surgical site 12 is arranged in the region of a focal plane 70 of a microscope unit 48. The surgical visualization system 10 may also be referred to as a surgical microscope.

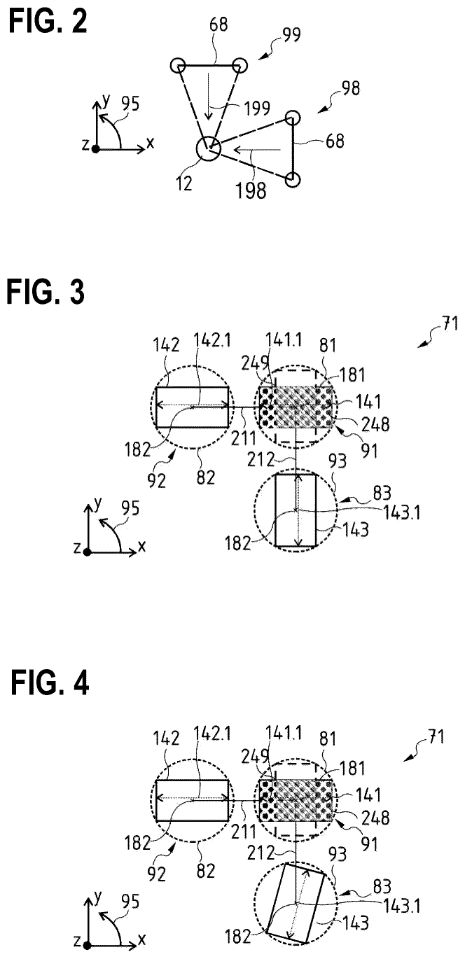

The surgical visualization system 10 includes the microscope unit 48, which is mounted on a movable arm 46. Said microscope unit includes a first optical channel 91, a second optical channel 92 and a third optical channel 93. FIG. 1 shows corresponding beam paths or beams 81, 82, 83 for each optical channel 91, 92, 93. The beams 81, 82, 83 are incident on the focal plane 70 at different polar angles 94, as a result of which the stereo impression arises when corresponding stereo pairs are formed (this will be explained in detail later in connection with FIG. 2 and FIG. 3 and FIG. 4).

In the example of FIG. 1, the beams 81, 82, 83 propagate jointly through a main objective 18 (the optical axis 20 of the main objective is represented by a dash-dotted line and runs parallel to the z-axis) and subsequently propagate through respective zoom systems 17 (afocal zoom systems 17) and imaging systems 16. Since each optical channel 91, 92, 93 includes a dedicated zoom system 17, the magnification factor can be set separately for each optical channel 91, 92, 93. In other words, the sizes of the fields of view of the various camera images captured with the cameras 41, 42, 43 can be set separately for each optical channel 91, 92, 93. The specific configuration of these units 16, 17, 18 is unimportant in terms of the techniques described herein: This is because the prior art has disclosed different optical configurations of the microscope unit 48. These approaches known from the prior art may be used for the techniques described herein. For example, FIG. 1 schematically shows a scenario in which the beam paths of the optical channels 91, 92, 93 run parallel to one another along a Z-axis, which is defined perpendicular to the focal plane 70 or parallel to the optical axis 20, over a relatively large region. However, conceivable variants include those in which at least one of the optical channels 91, 92, 93 defines a beam path which extends away from the beam paths of the two other optical channels, i.e. which is for example steered away from the Z-axis at a position downstream of the main objective 18 by way of a mirror. It would be conceivable in such a scenario for only two optical channels to provide a corresponding afocal zoom system, while the beam path steered away from the Z-axis does not include a zoom system.

Furthermore, it would be optionally possible for the surgical visualization system to include a stereo eyepiece for the first optical channel and the second optical channel and/or a mono eyepiece for the third optical channel. However, it would also be possible for the surgical visualization system to not include any eyepiece.

Moreover, the variant of FIG. 1 only allows joint focusing for all three optical channels 91, 92, 93. Setting the main objective 18 brings about the focusing. It would also be conceivable in other examples for different focusing, i.e. different focal planes 70, to be rendered possible for the various optical channels 91, 92, 93 by way of appropriate objective lenses.

FIG. 1 also shows that the observer 98 and a further observer 99 may each wear a head-mounted display apparatus 56 displaying stereoscopic images. While head-mounted display apparatuses 56 are used in FIG. 1, other types of display apparatuses may also be used in other variants, for example stereo screens or digital eyepieces. So-called BOOM (binocular omni-orientation monitor) systems are examples of digital eyepieces. It is also possible to use display apparatuses which allow for images captured with the microscope unit 48 to be overlaid on a view of the surroundings (from the perspective of the observer): such systems are often referred to as “virtual reality (VR)” systems.

FIG. 1 also shows the connecting line 68 between the respective right eye 60 and the respective left eye 62. The orientation of these connecting lines 68 in relation to the XY-plane defines the actual perspective of the respective observer 98, 99. The object is for the stereoscopic images to have an image perspective corresponding to this actual perspective. FIG. 2 initially illustrates aspects in connection with the actual perspective of the two observers 98, 99.

FIG. 2 illustrates a plan view of the surgical site 12, i.e. the Z-axis extends perpendicular to the plane of the drawing in FIG. 2. The arrangement of the perspectives 198, 199 for the observer 98 and the observer 99 is shown in relation to the surgical site 12. The two observers 98, 99 view the surgical site 12 from perspectives 198, 199 which have an azimuth angle offset 95 of approx. 90°.

FIG. 3 shows the beams 81, 82, 83 of the optical channels 91, 92, 93 in the plane 71 (cf. FIG. 1, the dash-dotted line therein). The positions of the chief rays 181, 182, 183 (crosses in FIG. 3) of the beams 81, 82, 83 in this case correspond to the polar angles at which the respective beams 81, 82, 83 are incident on the focal plane 70. The stereo impression is achieved by using different polar angles.

FIG. 3 also shows a projection of these beams 81, 82, 83 onto the camera sensors 141, 142, 143 of the cameras 41, 42, 43. FIG. 3 also plots the longitudinal axes 141.1, 142.1, 143.1 of the camera sensors 141, 142, 143 (dotted arrows).

It is evident from FIG. 3 that the longitudinal axis 141.1 of the camera sensor 141 runs parallel to the longitudinal axis 142.1 of the camera sensor 142 (these longitudinal axes extend in the left-right direction in FIG. 3). The longitudinal axis 141.1 of the camera sensor 141 and the longitudinal axis 142.1 of the camera sensor 142 both run parallel to the X-axis. It is also evident from FIG. 3 that the longitudinal axis 143.1 of the camera sensor 143 runs along the Y-axis and thus forms an angle of approx. 90° with the respective longitudinal axes 141.1,142.1 of the camera sensors 141, 142 (in the example of FIG. 3, the longitudinal axis of the camera sensor 143 extends in the up-down direction). This thus means that camera images captured with the cameras 41, 42, 43 have different native perspectives, which are given by the respective orientations of the longitudinal axes 141.1, 142.1, 143.1. For example, if the camera images are reproduced on display apparatuses with corresponding aspect ratios, then the native perspectives for the camera images captured with the cameras 41 complete 42 would be identical; and the native perspective for the camera images captured with camera 43 would be rotated through 90° (cf. FIG. 2).

The offset of the central ray 181 of the beam 81 from the central ray 182 of the beam 82 defines a stereo axis 211; the offset of the central ray 181 of the beam 81 from the central ray 183 of the beam 83 defines a stereo axis 212.

The stereo axis 211 and the stereo axis 212 have an azimuth angle offset of approx. 90° with respect to each other. The stereo axis 211 extends along the X-axis; and the stereo axis 212 extends along the Y-axis.

Thus, in the example of FIG. 3, the angle between the stereo axes 211, 212 is 90° and hence equal to the angle between the longitudinal axis of the camera sensor 141 and the longitudinal axis of the camera sensor 143 or equal to the angle between the longitudinal axis of the camera sensor 142 and the longitudinal axis of the camera sensor 143. However, it would in principle be conceivable for the longitudinal axis of the camera sensor 143 to form a different angle with the longitudinal axes of the camera sensors 141, 142 than the angle between the stereo axes 211, 212. Such a scenario is shown in FIG. 4, for example.

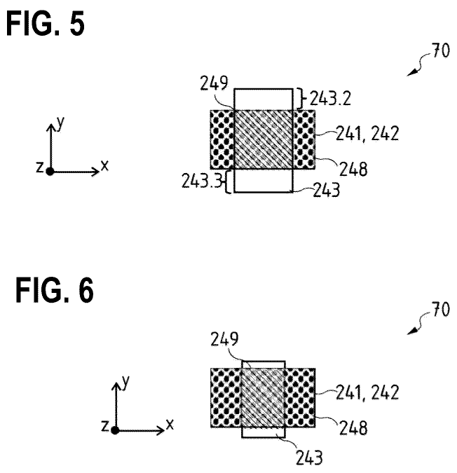

FIG. 5 schematically illustrates the regions 241, 242, 243 imaged onto the camera sensors 141, 142, 143 by the beam paths 91, 92, 93 for the scenario from FIG. 3. These regions 141, 142, 143 show the surgical site and are arranged in the focal plane 70 (see FIG. 1). The optical channel 91 images a region 241 in the focal plane 70 onto the camera sensor 141. The optical channel 92 images a region 242 in the focal plane 70 onto the camera sensor 142.

All regions 241, 242, 243 are rectangular and have the same aspect ratio; the extents of the regions 241, 242, 243 are also identical. The shape of the regions or the aspect ratio of the regions 241, 242, 243 is defined inter alia by the shape of the camera sensors 141, 142, 143 in this case. The sizes of the regions 241, 242, 243 are defined by a magnification factor of the respective zoom optical system 17. In principle, it is conceivable for the various regions 241, 242, 243 to have different sizes owing to the choice of different magnification factors: for example, cf. FIG. 6; FIG. 6 corresponds in principle to the scenario from FIG. 5, with a larger magnification factor having been chosen for the optical channel 93 such that the imaged region 243 has a smaller extent in the focal plane 70 than the regions 241, 242. Such a scenario enables better image resolution for structures arranged in the region 243. Such a technique is desirable in particular when the activity of the observer 98 is in essence concentrated on the centre of the regions 241, 242 such that it is acceptable for the field of view of the observer 99 to be significantly smaller than the field of view of the observer 99.

In principle, it is also conceivable for the shapes and/or the aspect ratios and/or the sizes of the various regions to deviate from one another. However, a variant according to FIG. 3 and FIG. 5 is discussed in detail below in order to illustrate the disclosure as simply as possible.

In the example of FIG. 3 or FIG. 5, the region 241 thus is congruent with the region 242, i.e. the region 241 and the region 242 have a full overlap. The corresponding stereo region 248 (dotted filling in FIG. 5) corresponds to both the region 141 and the region 242 on account of this full overlap. Moreover, FIG. 5 also illustrates the region 243 in the focal plane 70; this region is imaged onto the camera sensor 143 by the optical channel 93. FIG. 5 also illustrates the stereo region 249 (illustrated with hatching) which arises as a result of the overlap of the region 241 with the region 243. Further portions 243.2, 243.3 of the region 243 which are adjacent to the stereo region 249 are labelled in FIG. 5. No native stereo information is available for these portions 243.2, 243.3. The stereo region 249 is square (because the longitudinal axes 141.1, 143.1 of the camera sensors 141, 143 are oriented perpendicular to each other in the scenario of FIG. 3) and has a side length corresponding to the short side length of the rectangular stereo region 248. In the case of a 16:9 camera sensor, the stereo region 248 has a size ratio of 16:9 with respect to the stereo region 249, i.e. it is approx. 77% larger than the stereo region 259. In general, the ratio of the stereo region 248 to the stereo region 249 cannot be less than 150%. More generally, the ratio of the stereo region 248 to the stereo region 249 cannot be less than 200%, preferably not less than 150% and particularly preferably not less than 100%.

However, the size of the stereo region 249 may be adjustable in relation to the size of the stereo region 248, for example by setting the magnification factor.

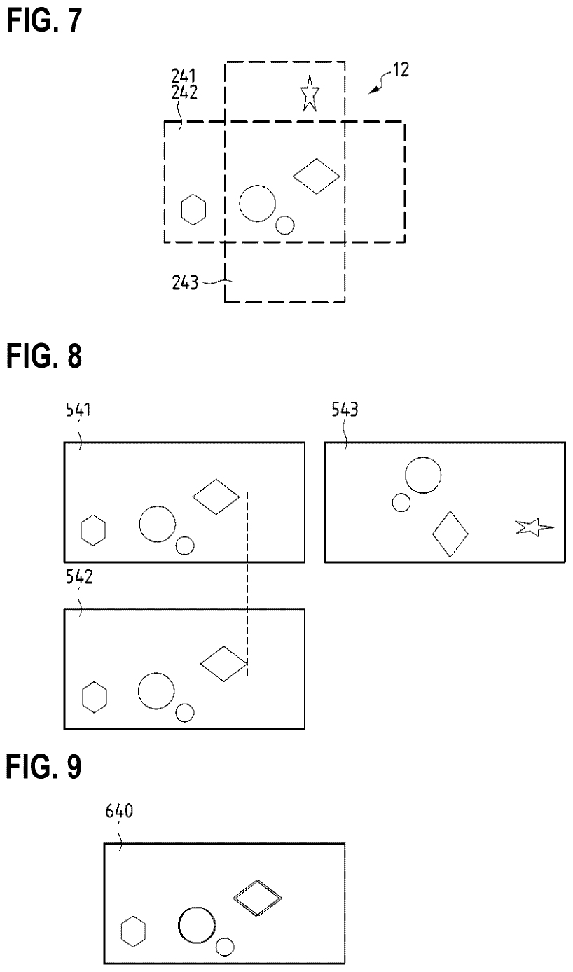

FIG. 7 illustrates the surgical site 12, in which a number of structures are arranged, by way of example. Moreover, the regions 241, 242, 243 are illustrated using dashed lines. FIG. 8 illustrates the camera image 541 of the surgical site 12 from FIG. 7, which is captured with the camera 41 of the optical channel 91. FIG. 8 also illustrates the camera image 543 of the surgical site 12, which is captured with the camera 43 of the optical channel 93. Since the camera images 541, 543 have different native perspectives of the surgical site 12, as discussed above in connection with FIG. 3, the various structures are rotated with respect to one another by 90° in the image plane.

FIG. 8 also illustrates the camera image 542. It is evident from a comparison of the camera image 541 with the camera image 542 that the various structures in the camera images 541, 542 have in part a lateral offset with respect to one another and parallel to the stereo axis 211 (cf. FIG. 3). This lateral offset becomes ever larger the farther the corresponding structure is situated away from the focal plane 70. By way of example, the lateral offset is emphasized in FIG. 8 for the diamond-shaped structure by way of the vertical dashed line.

A fully stereoscopic image 640 (cf. FIG. 9) for the observer 99 (cf. FIG. 1 and FIG. 2) can be generated by combining the camera images 541, 542. Since these camera images 541, 542 have the same native perspective of the surgical site FIG. 12 there is no need for further preprocessing of the camera images 541, 542: they may be superimposed directly as stereo channels. The image 640 is fully stereoscopic because it provides stereo information over its entire image field, i.e. in an image filling manner. The stereo region 248 corresponds to the regions 241, 242 of the surgical site 12 which are imaged by the two optical channels 91, 92 (cf. FIG. 5). This means that e.g. even those structures arranged right at the edge of the image field of the fully stereoscopic image 640 are displayed with depth information.

The generation of a partially stereoscopic image for the observer 98 (cf. FIG. 1 and FIG. 2) is discussed next. The partially stereoscopic image provides native depth information only in a partial region of the image field; outside of this partial region, structures are displayed either in monoscopic fashion or in synthetic-stereoscopic fashion.

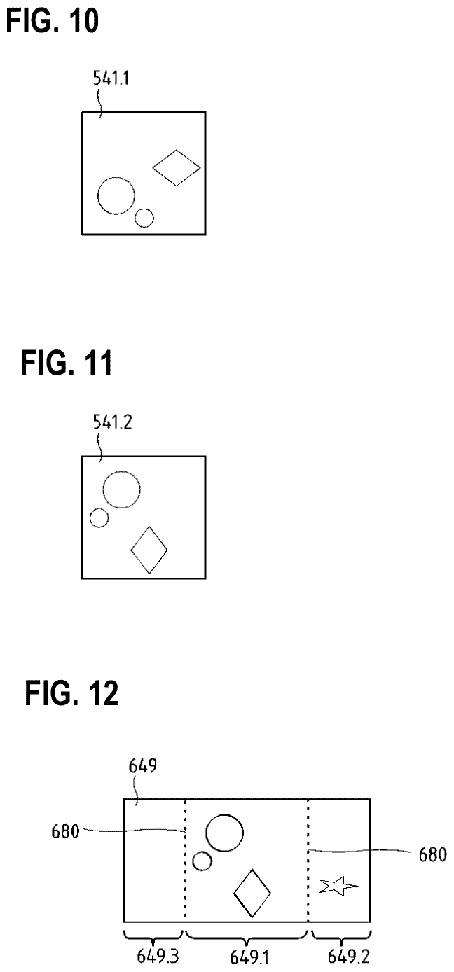

To generate the partially stereoscopic image, the portion of the image 541 which images the stereo region 249 (i.e. an overlap of the region 241 with the region 243, as illustrated in FIG. 5) is cut out of the image 541. The corresponding sectional image 541.1 is depicted in FIG. 10. The sectional image 541.1 is subsequently rotated, with the angle of rotation corresponding to the azimuth angle offset between the perspectives 198, 199 or the longitudinal axes 141.1, 143.1 (90° in the example of FIG. 3). The rotated sectional image 541.2 is depicted in FIG. 11. This rotated sectional image 541.2 may be used together with the camera image 543 in order to generate a partially stereoscopic image 649, cf. FIG. 12. The partially stereoscopic image 649 has a central portion 649.1; there the surgical site 12 is imaged with different stereo angles by the rotated sectional image 541.3 and the camera image 543. The central portion 649.1 shows the stereo region 249. Further portions 649.2, 649.3 are located adjacent to the central portion 649.1. The surgical site is not displayed in natively stereoscopic fashion there because the image information is available only from one polar angle with respect to the surgical site 12 or only from the camera image 543 (for example, this applies to the star-shaped structure in portion 649.2 of the partially stereoscopic image 649). The portions 649.2, 649.3 show the portions 243.2, 243.3 of the region 243 of the optical channel 93 which are adjacent to the stereo region 249 (cf. FIG. 5). For example, the portions 649.2, 649.3 could represent the respective region of the surgical site 12 in monoscopic or synthetic-stereoscopic fashion.

It is evident from a comparison of FIG. 9 and FIG. 12 that the field of view of the fully stereoscopic image 640 is the same as the field of view of the partially stereoscopic image 649. That is to say, the portion of the surgical site 12 imaged by the fully stereoscopic image 640 has the same area as the (other) portion of the surgical site 12 imaged by the partially stereoscopic image 649. This may however vary, for example according to the magnification factor used for the various optical channels (cf. FIG. 6).

Comparable fields of view being displayed allows the two observers 98, 99 to perceive a portion of the surgical site 12 of similar size. This allows a similar orientation for the observers 98, 99 or a comparable degree of detail. However, different perspectives are provided for the two observers 98, 99 at the same time, as discussed above.

For example, the display of the stereo region in the portion 649.1 of the partially stereoscopic image 649 could be graphically separated from the portions 649.2, 649.3. Corresponding transitions 680 between the portions 649.1, 649.2, 649.3 are shown in FIG. 12. For example, a frame could be displayed around the portion 649.1. Any other graphical indication could be provided at the transitions 680 between the portion 649.1 and the portions 649.2, 649.3. A separating line may be provided, for example with a specific separating line width. However, it would also be conceivable for the transition from the portion 649.1 to the portions 649.2, 649.3 to not be provided with a particular emphasis. For example, an image smoothing algorithm may ensure a smooth transition between the portions 649.1, 649.2, 649.3.

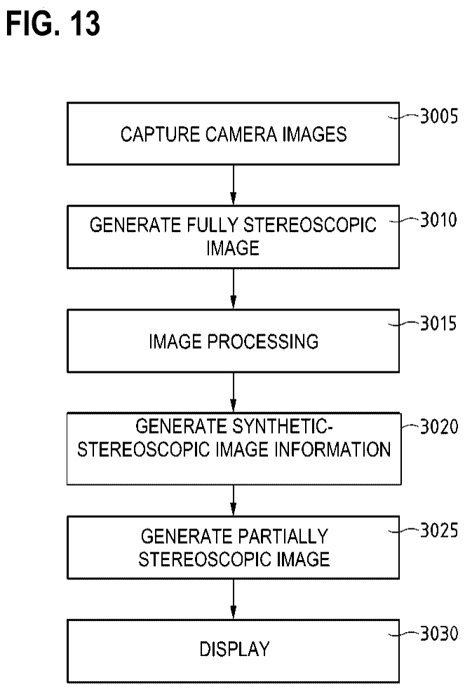

FIG. 13 is a flowchart of an exemplary method. The method of FIG. 13 may be executed by an electronic data processing device associated with a surgical visualization system. For example, the method of FIG. 13 may be executed by a processor when the processor loads and executes program code from a memory. For example, the processor may execute the techniques described herein in real time (e.g. 50 to 60 frames per second) and low latency (e.g. one to two frames latency).

Below, the method of FIG. 13 is discussed in the context of the surgical visualization system 10 of FIG. 1. The surgical visualization system 10 includes a total of three optical channels 91, 92, 93, which each include a dedicated camera 41, 42, 43. In this case, the camera 43 of one of the optical channels 93 includes a camera sensor 143 which is rotated with respect to the camera sensors 141, 142 of the other cameras 41, 42. By contrast, the two other camera sensors 141, 142 image the corresponding surgical site 12 with the same native perspective.

Camera images captured with the cameras 41, 42, 43 are obtained in Box 3005. To this end, the corresponding cameras 41, 42, 43 may be controlled by the electronic data processing device to capture the camera images. Corresponding image data may be received by way of a communications interface.

A fully stereoscopic image is generated in Box 3010 based on the camera images 541, 542 captured with the cameras 41, 42. For example, this may be implemented by overlaying the two camera images 541, 542 if they both natively have the same perspective of the surgical site 12 (corresponding techniques have been described above in connection with FIG. 9). The camera image 541 thus corresponds to the first stereo channel of the fully stereoscopic image, and the camera image 542 corresponds to the second stereo channel of the fully stereoscopic image.

Image processing of the camera image 541 is performed in Box 3015. In particular, that portion of the camera image 541 which images a stereo region 249 also imaged by the camera image 543 is cut out and rotated such that the perspective of the partial camera image obtained thus corresponds to the perspective of the camera image 543 (corresponding techniques have been discussed above in connection with FIG. 10 and FIG. 11).

Optionally, synthetic-stereoscopic image information may be generated in Box 3020 for certain regions of the surgical site 12 which are imaged by the camera image 543 and are arranged adjacent to the stereo region 249. For example, a machine learning model or any other algorithm could be used to this end. For example, such a model could obtain multiple camera images, for example the camera images 541, 542, 543, as an input. By virtue of a corresponding model obtaining not only the camera image 543 as input but also the camera images 541, 542, it is possible to use extended information about the surgical site 12 a better synthetic-stereoscopic reconstruction. For example, information about a current magnification factor of the microscope and/or a current working distance may be used. The quality of a corresponding reconstruction for generating synthetic-stereoscopic image information can be improved in this way.

Corresponding techniques are known in principle from the prior art and can be used here. For example, see Bartolomei, Luca, et al. “Stereo Anywhere: Robust Zero-Shot Deep Stereo Matching Even Where Either Stereo or Mono Fail.” arXiv preprint arXiv:2412.04472 (2024) or Gao, Kyle, et al. “Nerf: Neural radiance field in 3d vision, a comprehensive review.” arXiv preprint arXiv:2210.00379 (2022).

Then, the further stereoscopic image is generated in Box 3025. This is implemented based on the partial camera image from Box 3015 and, if present, the image information from Box 3020 and, in principle, based on the camera image 543 captured in Box 3005. For example, the further stereoscopic image in Box 3025 could be generated as a fully stereoscopic image. The image information of the camera image 543 outside the stereo region 249, for example, could be discarded to this end. It would also be conceivable for a magnification factor of the optical channel 93 to be chosen such that the entire image area of the camera image 543 is located within the camera image 541 such that the stereo region 249 is the same as the region 243 imaged by the camera image 543. However, the further stereoscopic image could also be generated as a partially stereoscopic image in Box 3025. To this end, a portion (cf. FIG. 12: portion 649.1) of the partially stereoscopic image may include native stereo information. This is the portion of the partially stereoscopic image which reproduces the stereo region 249. Another portion (cf. FIG. 12: portions 649.2, 649.3) of the partially stereoscopic image can reproduce monoscopic information or synthetic-stereoscopic information.

The first stereo channel of the further stereoscopic image is thus based on the partial camera image from Box 3015, and the second stereo channel of the partial stereoscopic image is based on the camera image 543.

Optionally, it would be possible to make use of a smoothing of transitions between the different portions of the partially stereoscopic image; corresponding techniques have been discussed in connection with the image smoothing algorithm and the transition 680 in FIG. 12.

In Box 3030, stereoscopic display devices are controlled to display the fully stereoscopic image from Box 3010 on the one hand and the partially stereoscopic image from Box 3025 on the other hand. This serves different observers who have different perspectives of the surgical site.

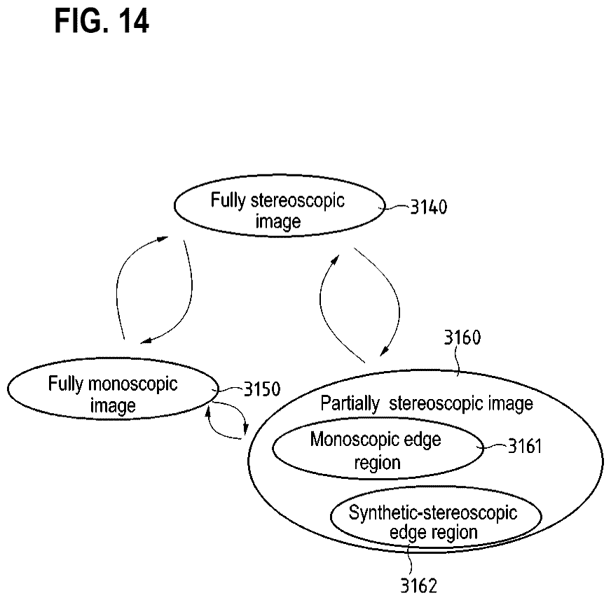

FIG. 14 illustrates various display modes 3140, 3150, 3160 in which a surgical visualization system, such as the surgical visualization system 10 (cf. FIG. 1) in particular, can be operated. In the example of FIG. 14, a partially stereoscopic image of the surgical site 12, for example the partially stereoscopic image 649, is displayed in the display mode 3160. It includes a portion 649.1 which stereoscopically displays a stereo region of the surgical site 12 and moreover includes one or more further portions 649.2, 649.3, which display one or more portions of the surgical site adjacent to the stereo region either in monoscopic fashion (display mode 3161) or else in synthetic-stereoscopic fashion (display mode 3162) or else completely block out these portions, e.g. fill them with a greyscale value. For example, the method according to FIG. 13 could be used in the display mode 3160.

Furthermore, other display modes 3140, 3150 are also depicted in FIG. 14. Thus, a fully stereoscopic image whose field of view is restricted to the stereo region 249 may be displayed instead of a partially stereoscopic image in the display mode 3140. Thus, the field of view of the fully stereoscopic image used in the display mode 3140 is typically smaller than the field of view of the partially stereoscopic image displayed in the display mode 3160 because those portions of the field of view of the corresponding camera image for which no stereoscopic information is available are discarded. Moreover, the aspect ratio of such a fully stereoscopic image is typically different from the aspect ratio of a stereoscopic display apparatus. This is because the camera sensors and the display apparatuses typically have an aspect ratio of 16:9. In such a case, a reproduction of a fully stereoscopic image representing only the stereo region 249 may have large “black bars” in the edge region of the stereoscopic display apparatus. For example, such a display mode 3140 might be activated by the user 99 if only image information from the centre of the field of view of the camera image 543 is required, where the stereo region 249 is located. The portions of the camera image 543 that image portions of the region 243 outside the stereo region 249 can be discarded in such a case.

A fully monoscopic image, for example corresponding to the camera image 543, may be displayed in the display mode 3150. In such a case, the camera image 543 need not be processed further but may be displayed directly. This may be preferred by the user 99 that the displayed image is not decomposed into different portions which display stereoscopic information on the one hand and monoscopic or synthetic-stereoscopic information on the other hand. It is for this reason that the user may activate the display mode 3150.

It would be conceivable for the user to be able to switch between the various display modes 3140, 3150, 3160. It would also be conceivable for certain display modes to be activated automatically. For example, the display mode 3140 could be activated automatically in a scenario, as described above in connection with FIG. 6, in which the stereo region 249 is relatively large in relation to the entire region 243 imaged by the camera image 543 (for example making up more than 80%).

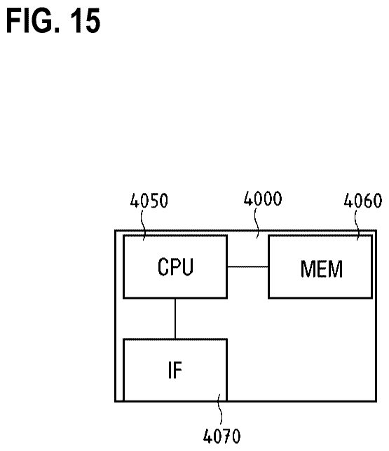

FIG. 15 schematically illustrates an electronic data processing device 4000. The latter includes a processor 4050 and a memory 4060 and a communications interface 4070. The processor 4050 can load program code from the memory 4060 and execute said program code. The processor 4050 can receive camera images or control one or more components of a surgical visualization system, for example the surgical visualization system 10 of FIG. 1, via the interface 4070. The processor 4050 can control one or more display devices by way of the interface 4070, for example in order to reproduce partially or fully stereoscopic images. The processor 4050 loading and executing program code from the memory 4060 causes the processor 4050 to execute techniques as described herein, for example in connection with the method of FIG. 13. For example, the processor 4050 may execute various techniques described herein for capturing and post-processing images in real-time with low latency. For example, the processor 4050 could process approx. 30 or more camera images per second, for example in the range between 40 and 60 camera images per second. The processing time required by the processor 4050 for processing the camera images may for example be less than 100 ms, optionally less than 50 ms, further optionally less than 20 ms and further optionally less than 1-2 ms. For example, the time period required for processing a camera image by the processor may correspond to that required for the capture of two further camera images or three further camera images.

It goes without saying that the features of the embodiments and aspects of the disclosure described above can be combined with one another. In particular, the features can be used not only in the combinations described but also in other combinations or on their own, without departing from the scope of the disclosure.

For example, techniques in which a fully stereoscopic image and a partially stereoscopic image are displayed have been described above. In this case, the fully stereoscopic image includes native stereo information over its entire image field, while the partially stereoscopic image provides monoscopic or synthetic-stereoscopic information in one or more portions. However, it would also be conceivable in other examples for two partially stereoscopic images to be generated and displayed with the three optical channels discussed above, with native-stereoscopic information being displayed in a larger portion of the one partially stereoscopic image, in comparison with the other partially stereoscopic image. This thus means that the one partially stereoscopic image images a corresponding stereo region in a larger portion of its image field than the other partially stereoscopic image.

A further variant consists of two fully stereoscopic images being displayed, with the first fully stereoscopic image however having a larger field of view than the second fully stereoscopic image. In other words, the fully stereoscopic image may stereoscopically display the first stereo region, and the second fully stereoscopic image may stereoscopically display the (smaller) second stereo region. Further image portions, which are located in the field of view of the third optical channel and for which no native stereo information is available, can be blocked out or represented by a greyscale value in that case.

Furthermore, techniques have been described above in which three optical channels are each equipped with their own zoom optical system. However, it would also be conceivable for only some or none of the optical channels to be equipped with a zoom optical system.

Furthermore, techniques have been described above in which at least one portion of that region of the focal plane which is imaged by the third optical channel 93 is located outside the stereo region, which is defined by the overlap of the region imaged by the third optical channel 93 with the region imaged by the first optical channel 91. However, it would for example also be conceivable for the magnification of the third optical channel 93 to be chosen in such a way that the entire region imaged by the third optical channel 93 in the focal plane 70 overlaps with the region imaged by the first optical channel 91. Two fully stereoscopic images can be displayed in that case.

1. A surgical visualization system, comprising:
a first optical channel which has a first camera and images a first region of a focal plane;
a second optical channel which has a second camera and images a second region of the focal plane;
a third optical channel which has a third camera and images a third region of the focal plane,
wherein the first optical channel and the second optical channel image a first stereo region, which is defined by the an overlap of the first region and the second region, in the focal plane in stereoscopic fashion,
wherein the first optical channel and the third optical channel image a second stereo region, which is defined by the overlap of the first region and the third region, in the focal plane in stereoscopic fashion,
an electronic data processing device configured to control at least one display apparatus for displaying a fully stereoscopic image and a partially stereoscopic image,
wherein the fully stereoscopic image represents the first stereo region in stereoscopic fashion, and
wherein the partially stereoscopic image represents the second stereo region in stereoscopic fashion and at least a portion of the third region adjacent to the second stereo region in monoscopic or synthetic-stereoscopic fashion.

2. The surgical visualization system according to claim 1, wherein a first stereo axis of the first stereo region forms an angle in the range from 85° to 95° with a second stereo axis of the second stereo region.

3. The surgical visualization system according to claim 2, wherein a first camera sensor of the first camera is rectangular with a first longitudinal axis,
wherein a second camera sensor of the second camera is rectangular with a second longitudinal axis,
wherein a third camera sensor of the third camera is rectangular with a third longitudinal axis,
wherein the first longitudinal axis and the third longitudinal axis form an angle between them which corresponds to the angle between the first stereo axis and the second stereo axis, and wherein the second longitudinal axis and the third longitudinal axis form an angle between them which corresponds to the angle between the first stereo axis and the second stereo axis.

4. The surgical visualization system according to claim 1, wherein a ratio of a dimension of a field of view of the fully stereoscopic image to a dimension of a field of view of the partially stereoscopic image is in the range from 95% to 105%.

5. The surgical visualization system according to claim 1, wherein a ratio of the first stereo region to the second stereo region is not less than 150%.

6. The surgical visualization system according to claim 1, wherein the first optical channel images the first region with a first magnification,
wherein the second optical channel images the second region with the first magnification, and
wherein the third optical channel images the third region with a second magnification which is larger than the first magnification.

7. The surgical visualization system according to claim 1, wherein the first region, the second region, and the third region are all rectangular and have the same aspect ratio.

8. The surgical visualization system according to claim 1, wherein the electronic data processing device is configured to control the at least one display apparatus to display the partially stereoscopic image when a first display mode is activated,
wherein the electronic data processing device is configured to control the at least one display apparatus to display a fully monoscopic image when a second display mode is activated, and
wherein the fully monoscopic image represents the entire third region in monoscopic fashion.

9. The surgical visualization system according to claim 1, wherein the electronic data processing device is configured to control the at least one display apparatus to display the partially stereoscopic image when a first display mode is activated,
wherein the electronic data processing device is configured to control the at least one display apparatus to display a further fully stereoscopic image when a third display mode is activated, and
wherein the further fully stereoscopic image exclusively represents the second stereo region in stereoscopic fashion.

10. The surgical visualization system according to claim 1, wherein the electronic data processing device is configured to switch between the monoscopic representation and the synthetic-stereoscopic representation of the at least one portion of the third region adjacent to the stereo region based on an activated mode of operation.

11. The surgical visualization system according to claim 1, wherein the synthetic-stereoscopic representation is generated using a model which receives camera images from the first camera, the second camera and the third camera as input.

12. The surgical visualization system according to claim 1, wherein the electronic data processing device is configured to graphically separate the a representation of the second stereo region from the representation of the an adjacent portion of the third region.

13. The surgical visualization system according to claim 1, wherein the electronic data processing device is configured to smooth a transition between a portion of the partially stereoscopic image representing the second stereo region and a portion of the partially stereoscopic image representing an adjacent portion of the third region with an image smoothing algorithm.

14. A method for operating an electronic data processing device associated with a surgical visualization system, the method comprising:
obtaining a first camera image from a first camera of a first optical channel of the surgical visualization system, wherein the first camera image images a first region of a focal plane from a first perspective,
obtaining a second camera image from a second camera of a second optical channel of the surgical visualization system, wherein the second camera image images a second region of the focal plane from the first perspective,
obtaining a third camera image from a third camera of a third optical channel of the surgical visualization system, wherein the third camera image images a third region of the focal plane from a second perspective,
generating a first stereoscopic image based on the first camera image as corresponding first stereo channel and based on the second camera image as corresponding second stereo channel,
cutting out and rotating a portion of the first camera image based on a difference between the first perspective and the second perspective in order to obtain a part camera image,
generating a second stereoscopic image based on the a partial camera image as corresponding first stereo channel and based on the third camera image as corresponding second stereo channel,
controlling a display device to display the first stereoscopic image, and
controlling a further display device to display the second stereoscopic image.

15. The method according to claim 14, wherein the first stereoscopic image is a fully stereoscopic image.

16. The method according to claim 14, wherein the second stereoscopic image is generated as a partially stereoscopic image or fully stereoscopic image, depending on an active display mode.

17. The method according to claim 14, wherein the first camera image images the first region with a first magnification,
wherein the second camera image images the second region with the first magnification, and
wherein the third camera image images the third region with a second magnification.

18. The method according to claim 17, wherein the second magnification is larger than the first magnification.

19. The method according to claim 14, wherein the second stereoscopic image is generated as a partially stereoscopic image, and
wherein the method further comprises:
smoothing or graphically emphasizing a transition between a portion of the further stereoscopic image representing native-stereoscopic information and a further portion of the further stereoscopic image representing monoscopic or synthetic-stereoscopic information.

20. The method according to claim 14, wherein the surgical visualization system is the surgical visualization system according to claim 1 comprises:
a first optical channel which has a first camera and images a first region of a focal plane;
a second optical channel which has a second camera and images a second region of the focal plane;
a third optical channel which has a third camera and images a third region of the focal plane,
wherein the first optical channel and the second optical channel image a first stereo region, which is defined by an overlap of the first region and the second region, in the focal plane in stereoscopic fashion,
wherein the first optical channel and the third optical channel image a second stereo region, which is defined by the overlap of the first region and the third region, in the focal plane in stereoscopic fashion,
an electronic data processing device configured to control at least one display apparatus for displaying a fully stereoscopic image and a partially stereoscopic image,
wherein the fully stereoscopic image represents the first stereo region in stereoscopic fashion, and
wherein the partially stereoscopic image represents the second stereo region in stereoscopic fashion and at least a portion of the third region adjacent to the second stereo region in monoscopic or synthetic-stereoscopic fashion.
